(19) United States
(12) Patent Application Publication
(10) Pub. No.: US 2014/0079809 A1
(43) Pub. Date: Mar. 20, 2014

(54) COMPOUNDS USEFUL FOR THE PREVENTION OR TREATMENT OF ACCOMMODATIVE ASTHENOPIA

(71) Applicant: Sigma-Tau Industrie Farmaceutiche Riunite S.p.A., Rome (IT)

(72) Inventors: Nicola PESCOSOLIDO, Rome (IT); Aleardo KOVERECH, Rome (IT)

(73) Assignee: Sigma-Tau Industrie Farmaceutiche Riunite S.p.A., Rome (IT)

(21) Appl. No.: 14/082,457

(22) Filed: Nov. 18, 2013

Related U.S. Application Data
(62) Division of application No. 13/002,366, filed on Feb. 28, 2011.

(51) Int. Cl. A61K 31/205; A61K 36/30; A61K 33/30; A61K 33/00; A61K 31/355; A61K 33/32

(52) U.S. Cl. CPC A61K 31/205; A61K 31/355; A61K 33/32; A61K 33/30; A61K 33/00; A61K 36/30
USPC 424/639; 514/554; 514/458; 424/641; 424/722; 424/725

(57) ABSTRACT

The use of L-carnitine, in combination with antioxidants such as vitamin E and inorganic elements such as manganese, zinc, sodium and potassium, for the preparation of a physiological supplement or medicament for ophthalmic use, for the prevention or treatment of accomodative asthenopia is disclosed.

### CROSS REFERENCE TO RELATED APPLICATIONS

This application is a Divisional of U.S. patent application Ser. No. 13/002,366 filed Feb. 28, 2011, which was a 371 of PCT/EP2009/057939 filed Jun. 25, 2009, which in turn claimed the benefit of EP Patent Application No. 08159676.9 filed on Jul. 4, 2008, the contents of each of which are incorporated herein by reference.

### FIELD OF THE INVENTION

The present invention relates to the use of L-carnitine for the preparation of a physiological supplement or medicament in the form of eye-drops useful for the prevention or treatment of accomodative asthenopia.

Accomodative asthenopia is also known as ocular fatigue syndrome.

### BACKGROUND OF THE INVENTION

Today accomodative asthenopia has become more and more common than it used to be in the past.

To define accommodative asthenopia, we may refer to the definition devised during the GILV, a study on the relation between vision and work [Med. Lav. 1993 July-August; 84(4); 324-31); Med Lav. 1993 November-December; 84(6); 502-4; Med Lav. 1994 March-April; 85(2); 179-82].

The reasons originating ocular fatigue are many, and in some cases they are not well identified even if factors that more frequently contribute are the inappropriate correction of refractive defects, the insufficiently lit working environment and the use of video screens.

Accomodative asthenopia is not only caused by factors related to the working environment and the use of computers and others technological apparatus, but also by some alterations to the visual apparatus such as chronic conjunctivitis and blepharitis; dry eye syndrome; corneal opacity; Keratoconus; cataract; aphakia and pseudophakia; severe refractive defects; degenerative retinopathy; maculopathy with central metamorphopsia; visual filed alterations.

Thus, refractive defects (myopia, astigmatism and hypermetropia) are not caused or made worse by the use of screens; on the other hand they may cause accomodative asthenopia should they not be corrected.

The symptoms of accomodative asthenopia can be summed up in three main classes: visual, ocular and general symptoms.
Visual symptoms include photophobia; reduced visual acuity; blurred vision; double vision; transitory myopization; transitory removal from the convergence point; appearance or increase of phorias; coloured halos.
Ocular symptoms include lachrymation; increased winking; itch; irritation; dryness; soreness; feeling of a foreign body; feeling of globe heaviness; pain; conjunctive reddening; lachrymal film quality/quantity alteration.
General symptoms include cefalea; asthenia; nausea; dyspepsia; vertigo; general tension; fatigued; sleepy; hazy; dull; bleary; vomitous; heavy; and painful.

Previous uses of carnitine in the opthalmological field are already known.

In WO07/03481 describes the use of L-carnitine for the treatment of corneal diseases.

U.S. Pat. No. 5,037,851 describes the use of acetyl L-carnitine for the treatment of cataracts.

U.S. Pat. Nos. 5,145,871 and 5,432,199 describe the use of acetyl D-carnitine for the treatment of glaucoma.

U.S. Pat. No. 5,883,127 describes the use of acetyl L-carnitine for the treatment of maculopathy and macular degeneration.

In J. Ocul. Pharmacol. 1994 Winter; 10(4):643-51, is reported that free carnitine and acid soluble acylcarnitines are present in various tissues of the rabbit eye and play an important role in those tissues of the eye where cells of a muscular nature are present and may represent, after esterification, an important energy reserve.

None of the above-cited patents or publications describes or suggests the use of L-carnitine for preventing or treating accomodative asthenopia or ocular fatigue syndrome.

To date are not available on the market ophthalmic drugs useful for preventing or treating accomodative asthenopia.

In the medical field there is still a strongly perceived need for the availability of therapeutic agents or physiological supplement useful for preventing or treating accomodative asthenopia.

It has now been found that L-carnitine o a salt thereof, is an useful agents for the preparation of a physiological supplement or medicament, in the form of eye-drops, for the prevention or treatment of accomodative asthenopia or ocular fatigue syndrome.

What is meant by pharmaceutically acceptable salt of L-carnitine is any salt of the latter with an acid that does not give rise to toxic or side effects.

These acids are well known to pharmacologists and to experts in pharmacy. Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

What is meant by pharmaceutically acceptable salt of L-carnitine is also a salt approved by the FDA and listed in the publication Int. J. of Pharm. 33 (1986), 201-217, which is incorporated herein by way of a reference.

### SUMMARY OF THE INVENTION

It is therefore one object of the present invention to provide a physiological supplement or medicament, in the form of eye-drops, comprising as active ingredient L-carnitine or a pharmaceutically acceptable salt thereof for the prevention or treatment of accomodative asthenopia or ocular fatigue syndrome.

The eye-drops of the invention may comprise antioxidants such as, for example, vitamin E, and one or more inorganic elements such as, for example, manganese, zinc, sodium or potassium.

The eye-drops of the invention may further comprise Aloe Vera as an anti-inflammatory agent in a concentration ranging from 0.05 to 5%. The optional presence of Aloe Vera in the eye drops of the invention does not increase the pharmacological activity of the composition of the invention.

The use of aloe vera in the ophthalmic field is described in U.S. Pat. No. 6,013,259.

The eye-drops of the invention have an osmolality in a range of about 200 to about 400 mOsmols/kg; preferred of about 250 to about 350 mOsmols/kg; most preferred 300 mOsmols/kg. The osmolality of the eye drops of the invention is due to the presence of L-carnitine, the presence of other elements is not relevant for the osmolality.

The amount of L-carnitine present in the eye-drops of the invention is from about 2.0% to about 4.0%, preferred is from about 2.5% to from about 3.5%, most preferred is 3.0%.

The eye-drops of the invention may further comprise other antioxidants, vitamins and/or inorganic elements; Borage oil; epithelializing and anti-angiogenic agents; humidifying agents; regulator of the cellular osmolality; antibiotics; antiviral and antifungal agents; and/or one or more alkanoyl L-carnitines selected from the group consisting of acetyl, propionyl, valeryl, isovaleryl, butyryl and isobutyryl L-carnitine or a salt thereof.

It is a further object of the present invention the use of L-carnitine for the prevention or treatment of accomodative asthenopia or ocular fatigue syndrome.

It is a further object of the present invention the use of L-carnitine for preparing a medicament, or a physiological supplement, for ophthalmic use for the prevention or treatment of accomodative asthenopia or ocular fatigue syndrome.

It is a further object of the present invention the use of L-carnitine in combination with antioxidants such as, for example, vitamin E and one or more inorganic elements, such as, for example, manganese, zinc, sodium or potassium,

in which:
L-carnitine is present at a dose of from about 2.0% to about 4.0%, preferably from 2.5% to 3.5%, most preferably is 3.0%;
vitamin E is present preferably at a dose of about 0.05 to about 1.0% by weight, and most preferably at a dose of about 0.2%;
manganese is present preferably at a dose of about 0.01 to about 0.1 mg/L, and most preferably at a dose of about 0.055 mg/L;
zinc is present preferably at a dose of about 0.5 to about 1.5 mg/L, and most preferably at a dose of about 1.05 mg/L;
sodium is present preferably at dose of about 5 to about 5000 mg/L, and most preferably at a dose of about 33 mg/L;
potassium is present preferably at a dose of about 1 to about 1000 mg/L, and most preferably at a dose of about 12 mg/L;

and the osmolality is in a range of about 200 to about 400 mOsmols/kg; preferably of about 250 to about 350 mOsmols/kg; most preferably 300 mOsmols/kg;

for preparing a medicament for the prevention or treatment of disturbances due to ocular fatigue syndrome or accommodative asthenopia; in which said accomodative asthenopia or ocular fatigue syndrome is characterized by the symptoms selected from the group comprising: fatigued, painful, hazy or bleary of the eyes and sleepiness, vomitous and painful; due to the use of computer display.

For purposes of the present invention, it will be understood by those of ordinary skill that the methods of treatment and use described herein are meant to include methods of treating human or animal eyes. Such methods include administering a medicament, for example, eye drops in accordance with the present invention, to a human or animal eye to provide medicinal benefit to the treated eye. The amount of the eye drops administered to the patient is generally described as an amount which is effect to treat, even temporarily and or symptomatically one or more of the conditions described herein. Thus the methods include administering 1 or more drops in the affected eye one or more times daily. The clinician of ordinary skill will, of course, be able to determine optimum dosing based on assessment of the clinical condition and strength of the ingredients included in the medicament.

Reference is made herein to medicaments in the form of eye drops. It should be understood that for purposes of the present invention that eye drops include solutions, suspensions, gels, creams and ointments intended for ophthalmic use.

The eye-drops according to the present invention may additionally contain further antioxidants, vitamins, Borage oil; epithelializing and anti-angiogenic agents; humidifying agents; inorganic elements; regulators of the cellular osmolality; antibiotics; anti-inflammatory agents, antiviral, antifungal agents, buffering agents, tonicity adjusting agents, preservatives, pH adjusting agents, components commonly found in artificial tears, such as one or more electrolytes, and the like and mixtures thereof. It will be further understood that all ingredients included in the medicament/physiological supplement of the present invention are preferably ophthalmically acceptable and can be chosen from materials which are conventionally employed in ophthalmic compositions.

The term “ophthalmically acceptable” with respect to a formulation, medicament, composition or ingredient herein means having no persistent detrimental effect on the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, medicaments, compositions and ingredients are those that cause no substantial detrimental effect, even of a transient nature.

Aside from the amounts for each of the ingredients described herein, it will be understood that the compositions will include amounts generally understood in the art as being effective concentrations for such ingredients and as readily apparent to those of ordinary skill.

The following examples illustrate the invention.

### EXAMPLE 1

30 healthy people, none of whom had any eye problems other than ametropia, 23-49 years old (average: 30.6) were enrolled for the study.

The subjects were randomly divided in two groups of 15 patients each.

A group of patients was treated ten days before the test with saline, the second group was treated for the same period with the eye drops having the following composition:
L-carnitine 3%
vitamin E 0.2%;
manganese 0.055 mg/L;
zinc 1.05 mg/L;
sodium 33 mg/L;
potassium 12 mg/L;
sodium mertiolate 0.02 mg/mL;
demineralized water;
volume 5 mL/vials;
osmolality of about 300 mOsmols/kg.

For the evaluation of visual fatigue a PC with a traditional video with a cathode ray tube display was used.

The distance between the subject and the display was 50 cm.

Front stimuli (figures, colour and lines) were presented to the subjects.

In the task, subjects had to indicate the position of the stimulus by pushing one of three keys on the keyboard. After the key was pressed, another set of stimuli was immediately presented.

All the subjects finished their tests within 40 minutes.

Subjective tests based on a questionnaire (60 items) provided various indices of visual fatigue after the above task.

Each question had five ranks, for example, from feeling no fatigue “1” to feeling very strong fatigue “5” when fatigue was asked about.

The conventional indices of visual fatigue were selected as fatigued; sleepy; hazy; dull; bleary; vomitous; heavy; and painful, which have conventionally been used for subjective tests of visual fatigue after viewing the display (VDT).

The results obtained are reported in the following Tables 1-8.

TABLE 1
FATIGUED

SCORE (1-5)

| PATIENT | CONTROL | TREATED |
|---|---|---|
| 1 | 4 | 3 |
| 2 | 3 | 2 |
| 3 | 4 | 3 |
| 4 | 5 | 4 |
| 5 | 3 | 2 |
| 6 | 5 | 3 |
| 7 | 4 | 3 |
| 8 | 3 | 4 |
| 9 | 3 | 2 |
| 10 | 4 | 3 |
| 11 | 5 | 3 |
| 12 | 5 | 2 |
| 13 | 4 | 4 |
| 14 | 4 | 3 |
| 15 | 3 | 4 |
| mean | 3.93 | 3.00 |
| sd | 0.80 | 0.76 |
| P< | | 0.01 |

TABLE 2
SLEEPY

SCORE (1-5)

| PATIENT | CONTROL | TREATED |
|---|---|---|
| 1 | 5 | 4 |
| 2 | 5 | 3 |
| 3 | 4 | 2 |
| 4 | 3 | 3 |
| 5 | 4 | 3 |
| 6 | 5 | 4 |
| 7 | 4 | 3 |
| 8 | 3 | 4 |
| 9 | 4 | 4 |
| 10 | 3 | 3 |
| 11 | 5 | 3 |
| 12 | 5 | 2 |
| 13 | 4 | 3 |
| 14 | 5 | 4 |
| 15 | 4 | 4 |
| mean | 4.20 | 3.27 |
| sd | 0.77 | 0.70 |
| P< | | 0.01 |

TABLE 3
HAZY

SCORE (1-5)

| PATIENT | CONTROL | TREATED |
|---|---|---|
| 1 | 3 | 2 |
| 2 | 3 | 3 |
| 3 | 4 | 3 |
| 4 | 4 | 2 |
| 5 | 5 | 4 |
| 6 | 4 | 2 |
| 7 | 5 | 3 |
| 8 | 3 | 3 |
| 9 | 4 | 2 |
| 10 | 3 | 2 |
| 11 | 5 | 4 |
| 12 | 4 | 4 |
| 13 | 5 | 2 |
| 14 | 3 | 2 |
| 15 | 4 | 2 |
| mean | 3.93 | 2.67 |
| sd | 080 | 0.82 |
| P< | | 0.001 |

TABLE 4
DULL

SCORE (1-5)

| PATIENT | CONTROL | TREATED |
|---|---|---|
| 1 | 5 | 4 |
| 2 | 4 | 3 |
| 3 | 3 | 2 |
| 4 | 4 | 3 |
| 5 | 5 | 4 |
| 6 | 4 | 2 |
| 7 | 5 | 3 |
| 8 | 4 | 4 |
| 9 | 5 | 4 |
| 10 | 5 | 2 |
| 11 | 4 | 3 |
| 12 | 5 | 4 |
| 13 | 5 | 3 |
| 14 | 3 | 3 |
| 15 | 4 | 4 |
| mean | 4.33 | 3.20 |
| sd | 0.72 | 0.77 |
| P< | | 0.001 |

TABLE 5
BLEARY

SCORE (1-5)

| PATIENT | CONTROL | TREATED |
|---|---|---|
| 1 | 5 | 3 |
| 2 | 3 | 4 |
| 3 | 4 | 2 |
| 4 | 4 | 2 |
| 5 | 4 | 4 |
| 6 | 5 | 3 |
| 7 | 3 | 2 |
| 8 | 4 | 3 |
| 9 | 3 | 2 |
| 10 | 5 | 4 |
| 11 | 4 | 3 |
| 12 | 5 | 2 |
| 13 | 3 | 3 |
| 14 | 3 | 4 |
| 15 | 4 | 3 |
| mean | 3.93 | 2.93 |
| sd | 0.80 | 0.80 |
| P< | | 0.01 |

TABLE 6
VOMITOUS

SCORE (1-5)

| PATIENT | CONTROL | TREATED |
|---|---|---|
| 1 | 1 | 1 |
| 2 | 1 | 1 |
| 3 | 1 | 1 |
| 4 | 2 | 2 |
| 5 | 1 | 1 |
| 6 | 2 | 1 |
| 7 | 2 | 1 |
| 8 | 1 | 1 |
| 9 | 1 | 1 |
| 10 | 2 | 1 |
| 11 | 1 | 1 |
| 12 | 2 | 1 |
| 13 | 2 | 1 |
| 14 | 1 | 1 |
| 15 | 2 | 1 |
| mean | 1.47 | 1.06 |
| sd | 0.52 | 0.26 |
| P< | | 0.05 |

TABLE 7
HEAVY

SCORE (1-5)

| PATIENT | CONTROL | TREATED |
|---|---|---|
| 1 | 2 | 1 |
| 2 | 2 | 1 |
| 3 | 1 | 2 |
| 4 | 2 | 1 |
| 5 | 1 | 1 |
| 6 | 1 | 2 |
| 7 | 2 | 1 |
| 8 | 1 | 1 |
| 9 | 1 | 1 |
| 10 | 2 | 1 |
| 11 | 2 | 1 |
| 12 | 2 | 1 |
| 13 | 1 | 1 |
| 14 | 2 | 1 |
| 15 | 1 | 1 |
| mean | 1.53 | 1.13 |
| sd | 0.52 | 0.35 |
| P< | | 0.05 |

TABLE 8
PAINFUL

SCORE (1-5)

| PATIENT | CONTROL | TREATED |
|---|---|---|
| 1 | 2 | 1 |
| 2 | 2 | 2 |
| 3 | 1 | 1 |
| 4 | 2 | 1 |
| 5 | 3 | 1 |
| 6 | 1 | 1 |
| 7 | 1 | 1 |
| 8 | 2 | 1 |
| 9 | 2 | 2 |
| 10 | 1 | 1 |
| 11 | 2 | 1 |
| 12 | 1 | 1 |
| 13 | 1 | 1 |
| 14 | 2 | 1 |
| 15 | 1 | 1 |
| mean | 1.60 | 1.13 |
| sd | 0.63 | 0.35 |
| P< | | 0.05 |

The results obtained indicate that the eye drops according to the invention reduced in a statistically significant manner the symptoms scored.

L-carnitine and its alkanoyl derivatives are known compounds, the preparation process for which is described in U.S. Pat. No. 4,254,053.

The physiological supplement or medicament according to the present invention may be bought with or without medical prescription.

The physiological supplement or medicament according to the present invention are composed of active ingredients which are familiar to operators in the medical field and already in use in clinical practice, and their pharmacotoxicological profiles are known.

Their procurement therefore is very easy, inasmuch as these are products which have been on the market now for a long time and are of a grade suitable for human or animal administration.

In the following are reported non limiting examples of compositions according to the present invention.

Eye-Drops 1
L-carnitine 2.7%
vitamin E 0.2%;
manganese 0.055 mg/L;
zinc 1.05 mg/L;
sodium 33 mg/L;
potassium 12 mg/L;
sodium mertiolate 0.02 mg/mL;
demineralized water;
volume 5 mL/vials.
   Osmolality of about 270 mOsmols/kg.

Eye-Drops 2
L-carnitine 3%
vitamin E 0.2%;
manganese 0.055 mg/L;
zinc 1.05 mg/L;
sodium 33 mg/L;
potassium 12 mg/L;
sodium mertiolate 0.02 mg/mL;
demineralized water;
volume 5 mL/vials.
   Osmolality of about 300 mOsmols/kg.

Eye-Drops 3
L-carnitine 3.3%
vitamin E 0.2%;
manganese 0.055 mg/L;
zinc 1.05 mg/L;
sodium 33 mg/L;
potassium 12 mg/L;
sodium mertiolate 0.02 mg/mL;
demineralized water;
volume 5 mL/vials.
   Osmolality of about 330 mOsmols/kg.

The compositions of the invention may further contain different preservatives and optionally further regulators of the osmolality, if any.

1. A method for preventing or treating accomodative asthenopia or ocular fatigue syndrome, comprising administering to a patient in need thereof eye drops comprising an effective amount of L-carnitine or a pharmaceutically acceptable salt thereof.

2. Method according to claim 1, wherein the L-carnitine is in combination with antioxidants and inorganic elements.

3. Method according to claim 2, wherein the antioxidant is Vitamin E and the inorganic elements are selected from the group consisting of manganese, zinc, sodium and potassium.

4. Method according to claim 1, wherein said accomodative asthenopia or ocular fatigue syndrome is characterized by symptoms selected from the group consisting of: fatigued, painful, hazy or bleary of the eyes, sleepiness and vomitous.

5. Method according to claim 1, wherein said accomodative asthenopia or ocular fatigue syndrome is due to the use of a computer display.

6. Method according to claim 1, wherein the eye drops comprise:
2.0% to 4.0% L-carnitine;
0.05 to 1.0% Vitamin E;
0.01 to 0.1 mg/L Manganese;
0.5 to 1.5 mg/L Zinc;
5 to 5000 mg/L Sodium; and
1 to 1000 mg/L Potassium.

7. Method according to claim 1, in which the pharmaceutically acceptable salt of L-carnitine is selected from the group consisting of chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

8. The method according to claim 1, wherein said eye drops further comprise: vitamins; Borage oil; epithelializing and anti-angiogenic agents; humidifying agents; antiinflammatory agents, regulator of the cellular osmolality; antibiotics; antiviral and antifungal agents; or one or more alkanoyl L-carnitines selected from the group consisting of acetyl, propionyl, valeryl, isovaleryl, butyryl and isobutyryl L-carnitine; and one or more excipients or diluents which are ophthalmologically acceptable.

9. The method according to claim 1, wherein the eye-drops have an osmolality in a range of about 200 to about 400 mOsmols/kg.

10. The method according to claim 9, wherein the eye-drops have an osmolality in a range of about 250 to about 350 mOsmols/kg.

11. The method according to claim 10, wherein the eye-drops have an osmolality of 300 mOsmols/kg.

12. The method according to claim 1, wherein the amount of L-carnitine present in the eye-drops is from about 2.0% to about 4.0%.

13. The method according to claim 12, wherein the amount of L-carnitine present in the eye-drops is from about 2.5% to from about 3.5%.

14. The method according to claim 13, wherein the amount of L-carnitine present in the eye-drops is 3.0%.

15. The method according to claim 1, wherein the eye drops comprise:
2.7% L-carnitine; 0.2% vitamin E; 0.055 mg/L manganese; 1.05 mg/L zinc; 33 mg/L sodium; 12 mg/L potassium; 0.02 mg/mL sodium mertiolate; and demineralized water; and
the eye drops have an osmolality of about 270 mOsmols/kg.

16. The method according to claim 1, wherein the eye drops comprise:
3% L-carnitine; 0.2% vitamin E; 0.055 mg/L manganese; 1.05 mg/L zinc; 33 mg/L sodium; 12 mg/L potassium; 0.02 mg/mL sodium mertiolate; and demineralized water; and
the eye drops have an osmolality of about 300 mOsmols/kg.

17. The method according to claim 1, wherein the eye drops comprise:
3.3% L-carnitine; 0.2% vitamin E; 0.055 mg/L manganese; 1.05 mg/L zinc; 33 mg/L sodium; 12 mg/L potassium; 0.02 mg/mL sodium mertiolate; and demineralized water; and
the eye drops have an osmolality of about 330 mOsmols/kg.
